(19) United States
(12) Patent Application Publication
(10) Pub. No.: US 2026/0123940 A1
(43) Pub. Date: May 7, 2026

(54) ORTHOPEDIC BONE AWL WITH OPTOELECTRONIC FEEDBACK

(71) Applicant: Waypoint Orthopedics, Inc., Malvern, PA (US)

(72) Inventors: Stephen Banco, Wyomissing, PA (US); Richard Briganti, Bala Cynwyd, PA (US); Lawrence Husick, Malvern, PA (US); Jeffrey O'Donnell, Malvern, PA (US)

(73) Assignee: Waypoint Orthopedics, Inc., Malvern, PA (US)

(21) Appl. No.: 19/117,929

(22) PCT Filed: Oct. 4, 2022
(86) PCT No.: PCT/US2022/077502

§ 371 Date: Apr. 2, 2025

(51) Int. Cl. A61B 17/16; A61B 34/30; A61B 90/00; A61B 90/30

(52) U.S. Cl. CPC A61B 17/1671; A61B 17/1626; A61B 34/30; A61B 90/06; A61B 90/30; A61B 2090/064; A61B 2090/306

(57) ABSTRACT

A tool having a tip for creating a bore in bone for the insertion of a pedicle screw or the like, which transmits the color of the tissue adjacent the tip of the tool, and a method of using the tool.

### BACKGROUND OF THE INVENTION

### 1. Field of Invention

The present invention relates generally to apparatus for inserting pedicle screws into the spine, and particularly to several embodiments of device whereby the likelihood of nerve damage caused by improperly placed pedicle screws can be reduced.

### 2. Discussion of the Prior Art

Instances arise when it becomes necessary to stabilize or fuse a portion of the spine from motion such as, for example, (1) after decompression wherein certain posterior spinal elements are removed to relieve pressure on neural elements, (2) after trauma, or (3) because of the presence of tumors. Instruments that accomplish spinal fixation are known in the form of pedicle screws which are adapted to be inserted in selected vertebrae, and stiff rods, plates, tethers and other devices that connect adjacent pedicle screw heads to one another after the screws are inserted, thus resulting in the fixing or bracing of all vertebrae spanned by the stabilizing apparatus.

The pedicles are the strongest parts of the spinal vertebrae and thus provide a secure foundation for the screws to which fixing rods or plates are attached. See R. Roy-Camille, et al, Internal Fixation of the Lumbar Spine with Pedicle Screw Plating, Clinical Orthopedics (February 1986), at page 7; and H. N. Herkowitz, et al, Instrumentation of the Lumbar Spine for Degenerative Disorders, Operative Techniques in Orthopaedics (January 1991), at page 91.

To achieve the greatest mechanical integrity when anchoring pedicle screws in a spine, it is therefore essential that the screws be guided and threaded in alignment with the pedicle axis and not be allowed to deviate off axis in which case the screw body or its threads will either break through the vertebral cortex and impinge on or become dangerously close to surrounding nerve roots, or merely be inserted into softer bone regions having less structural strength. A jig adapted for providing locations on the pedicles of a vertebra for insertion of pedicle screws is disclosed in U.S. Pat. No. 4,907,577 (Mar. 13, 1990) which discloses that the vertebral bodies will be fixed more stably the deeper the screws are inserted in the pedicle, and that slight deviations in the angle of screw insertion can injure nerve roots or the spinal cord.

Much appears in the literature with respect to the problems of misalignment of pedicle screws and the symptoms arising when the screws make contact with neural elements after breaking outside the pedicle cortex. Cutting into a nerve root or simply contacting the root gives rise to various postoperative symptoms such as dropped foot, neurological lesions, sensory deficits, or pain. The Adult Spine—Principles and Practice, Vol. II, at pages 1937 and 2035-36 (Raven Press 1991); J. L. West, et al, Complications of the Variable Screw Plate Pedicle Screw Fixation, Spine (May 1991), at 576-79; and J. L. West, et al, Results of Spinal Arthrodesis with Pedicle Screw-Plate Fixation, Journal of Bone and Joint Surgery (Sep. 1991), at 1182-83.

The prior art teaches the use of electrical stimulation as a means of estimating how near the tip of a bone awl is approaching a nerve root or the spinal cord. U.S. Pat. No. 5,474,558 is one such disclosure. The prior art also teaches the use of force sensors to estimate the position of the tip of a bone awl.

Conventional practice calls for the use of recognized landmarks along the spinal column for purposes of locating pedicle entry points, and the use of X-ray exposures or fluoroscopy to monitor the advancement of a metallic pedicle screw through the vertebra. But prolonged radiation exposure of the patient for purposes of proper screw placement is of course undesirable and this technique still has a significant misplacement rate. Further, prolonged and repeated exposure to ionizing radiation on the part of the surgeon and other medical personnel is undesirable and should be minimized whenever possible.

The prior art also teaches the use of a probe inserted by a surgeon into a bore in the vertebral body to “feel” the depth of penetration and path of the bore. The use of a probe, however, requires that the bore be partially formed, the tool used to form the bore withdrawn, the probe inserted and manipulated, then withdrawn, and the tool reinserted to either deepen the bore or to correct the trajectory of the bore prior to deepening. This repetitive process lengthens the time required to properly insert each pedicle screw, and thus, lengthens the entire procedure, potentially elevating the risk to the patient due to additional time under anesthesia, and time in on the operating table, in general.

It is known generally that various tissues of the body appear differently colored when observed under ambient light, as, for example, during open surgery or during autopsy. In particular, neural tissues such as the spinal cord and nerves emanating from the cord at the nerve roots are perceived under white light as translucent white. Cortical bone is perceived under white light as opaque white, while cancellous bone is perceived as a deep red color due to the high concentration of red blood cells that it contains. In a surgical setting, however, insertion of the pedicle screw is done “blind” (i.e., inside the bone, in a region where there is no ambient light and no way for the surgeon to visualize the tissues.)

The several embodiments of the present invention uses a light source and light sensor integrated into a bone awl to allow visualization of the color of the tissue adjacent the dip of the awl for determining if a pedicle screw to be inserted in the patient's spine might impinge on or come in dangerously close proximity to a nerve root or the spinal cord, as well as to visualize other tissues, particularly types of bone being penetrated by the tool itself.

### SUMMARY OF THE INVENTION

The several embodiments of the present invention overcome the above and other shortcomings in the prior art with respect to the location, placement and insertion of pedicle screws as part of a spine fixation.

The disclosed embodiments of the invention enable a surgeon to know, in situ, and prior to insertion, if a pedicle screw may (1) penetrate the cortex of a pedicle, (2) touch a nerve root, or (3) come in such close proximity to a nerve root or the spinal cord so as to require repositioning.

The disclosed embodiments of the invention enable screws to be inserted into a patient's bone tissue safely and without the requirement of continuous or frequent radiation exposure to ensure proper screw member alignment.

The disclosed embodiments of the invention reduce instances of postoperative complications such as dropped foot, neurological lesions, sensory deficits, or pain following surgery involving placement of screws in a patient's spine.

The disclosed embodiments of the present invention comprise a surgical tool for forming an opening in bone tissue for insertion of a screw and a display and control to permit operation of the surgical tool. The tool comprises a tool handle and a shaft extending from the handle. The shaft has a tip adapted to work an opening in the bone tissue. Contained within the tool is a light source, an illuminating optical fiber, an observing optical fiber, a light sensor, a microcontroller, a radio frequency transceiver, and a power source. The display and control is preferably implemented in computer application software operating on a tablet computer or smart phone that is able to communicate with the surgical tool using radio frequency signals such as Bluetooth. Optionally, various strain gauges and/or force sensors may be incorporated into the tool for transducing compression force applied to tissues, torques applied, and/or lateral forces applied, since various tissues exhibit differing structural characteristics. The signals transduced by these may be displayed using the computer application software on the tablet computer.

For a better understanding of the present invention, reference is made to the following description taken in conjunction with the accompanying drawings, and the scope of the invention will be detailed in the appended claims.

### BRIEF DESCRIPTION OF THE DRAWINGS

FIG. 1 depicts an exploded view of one embodiment of the instrument according to the present invention.

FIG. 2 depicts a block diagram showing the operational components of a preferred embodiment of the instrument according to the present invention;

FIG. 3A-F is a depiction of the user interface implemented in application software operating on a tablet computer or similar general purpose digital computing device;

FIG. 4 depicts in cross section a typical human vertebral body, showing a preferred embodiment of the instrument of the present invention forming a bore in the desired location for insertion of a pedicle screw, and showing as well the opposing path desired for insertion of another pedicle screw;

FIG. 5A depicts alternative embodiments of the shaft and tip of the instrument of the present invention, while FIG. 5B depicts the shaft of the preferred embodiment in cross-section; and

FIG. 6A and FIG. 6B depict in various aspects embodiments of the proximal and distal (top and bottom) handle shells of the instrument of the present invention.

### DETAILED DESCRIPTION OF THE INVENTION

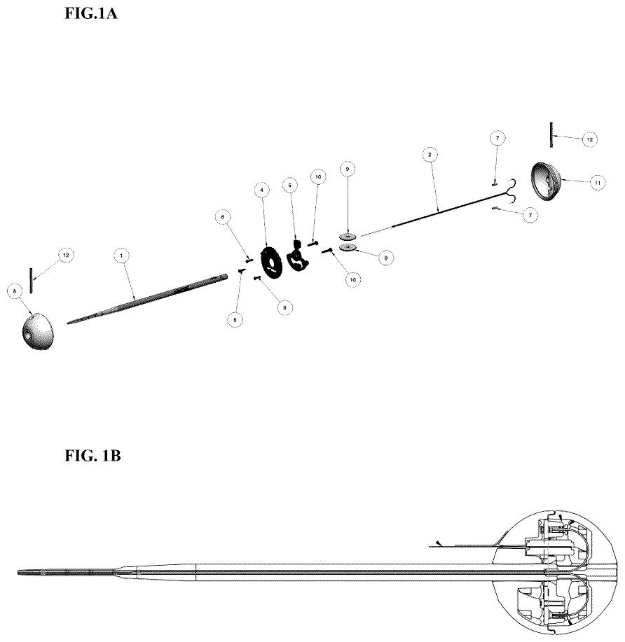

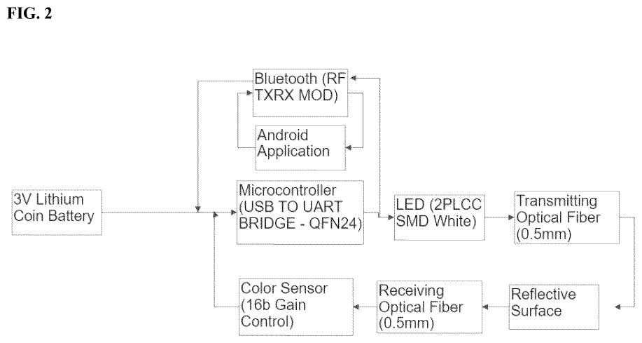

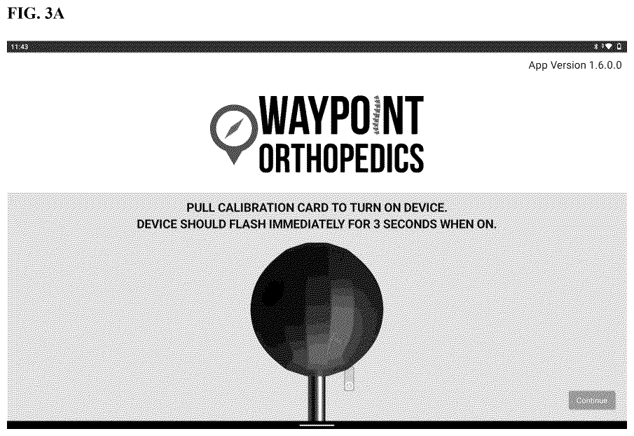

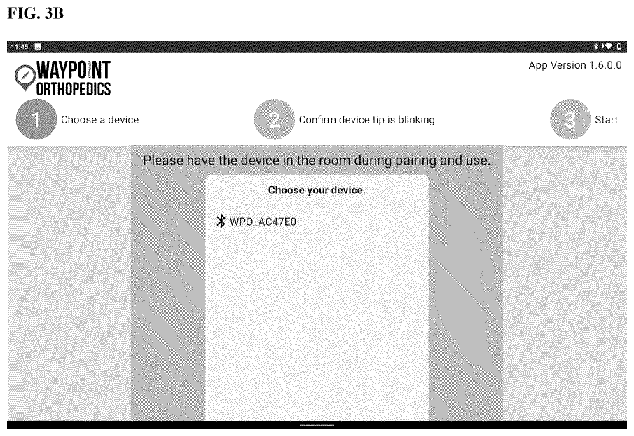

FIGS. 1A and 1B depict an exploded view, and a cross-sectional view, respectively, of an embodiment of the instrument according to the present invention. Element 1 is the awl shaft, which extends from the tip at the distal end, to the handle at the proximal end. Element 2 depicts the fiber optic pair which is carried by the shaft, and is comprised of an illumination fiber and a reflected light fiber. Element 3 is cement, preferably epoxy-type. Element 4 is a printed circuit board, which is described in greater detail in connection with the block diagram of FIG. 2. Element 5 is an optical alignment element that mates each of the optical fibers to the illumination source (not shown), and the light detector color sensor (not shown), respectively. Elements 6 are the fasteners that maintain the printed circuit board and the optical alignment element in relation to each other. Element 7 is a ferrule. Element 8 is the distal or top handle shell. Element 9 is a power source, preferably a pair of coin cell batteries. Element 10 is another fastener. Element 11 is the proximal or bottom handle shell. Element 12 is a dowel pin.

FIG. 2 is a block diagram showing the operating components of the instrument according to a preferred embodiment of the present invention. A lithium coin battery providing sufficient voltage and current provides power to the circuit, which is comprised of a universal asynchronous receiver-transmitter (UART), in communication with a Bluetooth transceiver, an LED white light source, and a light detector color sensor. Each of these elements is controlled by application software which may be, for example, an Android application running on a tablet computer or similar device. Light emitted by the LED white light source is directed through an illuminating optical fiber running from the handle of the instrument to the tip of the instrument where it exits the fiber and illuminates the area directly ahead of the tip. A reflected light optical fiber is disposed at the tip and runs to the handle where it is mounted to efficiently illuminate the light detector color sensor. Light reflected from tissues adjacent the instrument tip is directed up the reflected light optical fiber. The output of the light detector color sensor represents, in digital form, the color of the tissue adjacent the instrument tip. This digital information is transmitted via the Bluetooth transceiver to the tablet device, and is used in the application software to display the transduced color on the tablet for viewing by the surgeon.

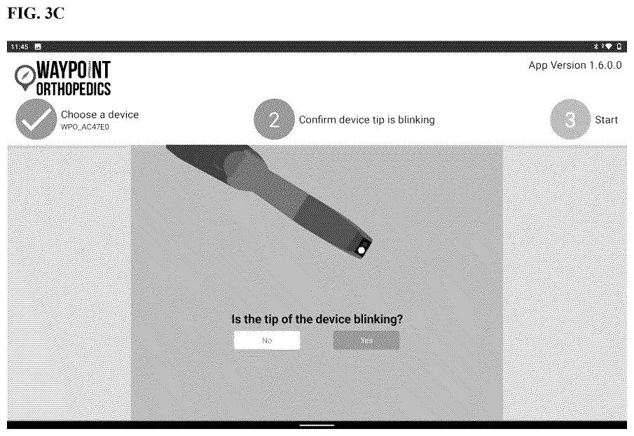

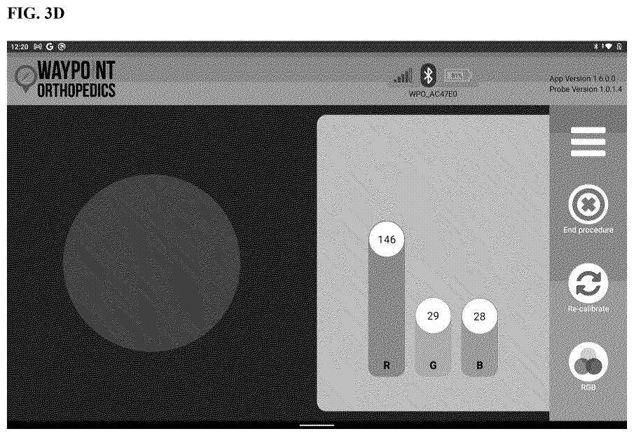

FIGS. 3A-F are black and white representations of the user interface, which in the preferred embodiment are displayed in color (but due to the rules of various patent offices, must be rendered here in black and white). The actual color of the tissue at the tip of the tool is shown in the user interface for the information of the surgeon performing a procedure.

When performing a pedicle screw insertion, a surgeon follows a Device Operational Sequence as follows:
1) Power up the circuit within the instrument
   a. The instrument may be provided with a switch, or the power source, such as a battery, may be physically isolated from the circuit, and the user may remove or dislodge the isolating element, e.g., a plastic insulator tab or similar device.) This step may alternatively be prompted on screen after the user interface application software has been launched on the user interface device.
2) Turn on user interface device (android, iOS or similar table computer or smart phone, equipped with user interface software according to the present invention.)
3) Launch user interface application software on the user interface device.
4) The user interface application will use the available Bluetooth transceiver to search for an instrument within range, and finding one, will prompt the surgeon to connect to the instrument. The identity of the instrument will be recorded, and if required, a calibration process may be executed. 5) The surgeon will be prompted to enter information identifying the patient to undergo the procedure.
6) The surgeon is the able to use the instrument to create bores into the vertebrae. The color of the tissue adjacent the tip of the instrument is displayed on the screen of the user interface device in near-real-time, allowing the surgeon to visualize the tissue about to be penetrated by the instrument. This color information is stored by the user interface software application for future review. In addition, ancillary data such as real time, exerted forces, and the like may be displayed and recorded, if available.
7) At the conclusion of the surgical procedure, the surgeon is prompted to disconnect the instrument from the user interface software. Upon disconnection the instrument is rendered unusable, and the surgeon is reminded to properly dispose of the instrument as infectious medical waste.

The preferred sensor for use in the preferred embodiment of the present invention is the TCS3472 color sensor from AMS, AG. The preferred light source for use in the present invention is the STW9C2PB-S from Seoul Semiconductor. Of course, those skilled in the art may recognize that many other similar sensor and LED light sources may be employed without departing from the functional requirements of the invention.

### EXAMPLE

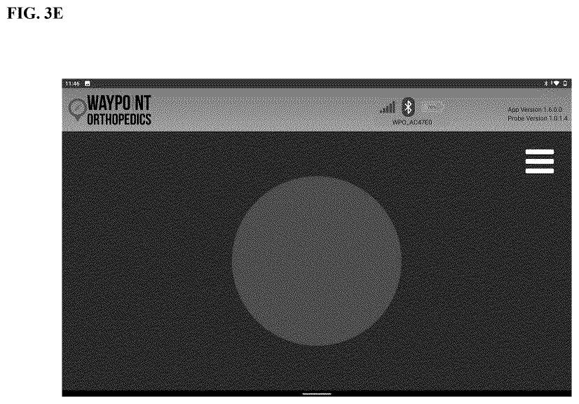

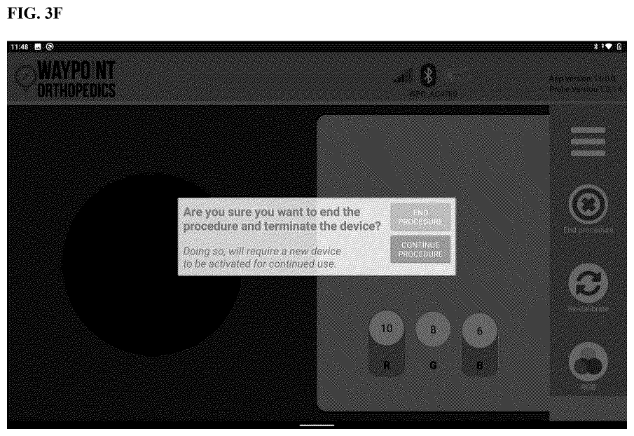

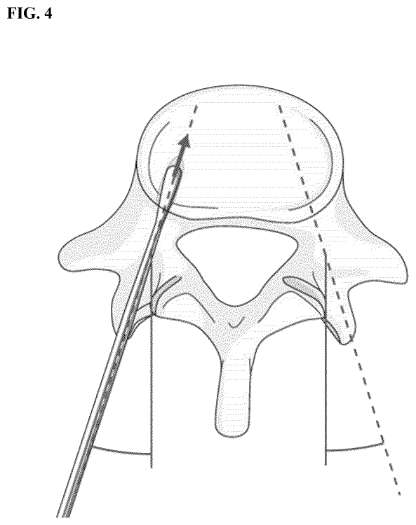

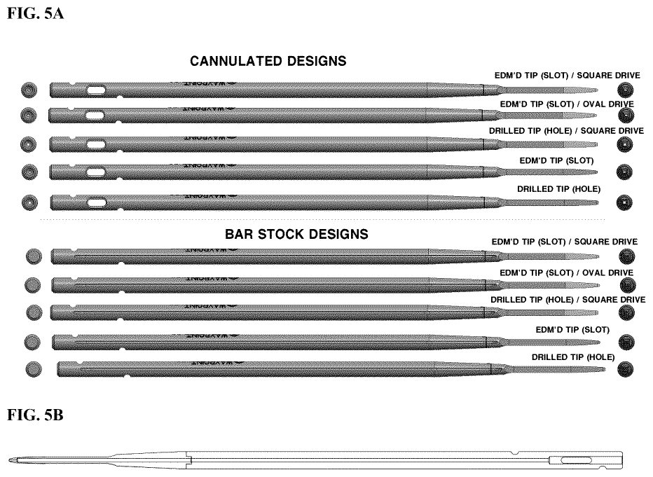

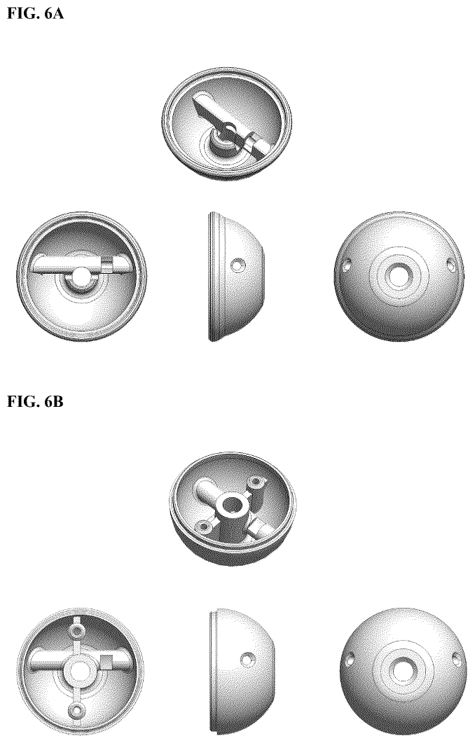

The surgical procedure envisioned is similar to those of the prior art but is informed by the sensor and display provided as part of the present invention. After conventional lumbar operational site preparation, a surgeon determines an entrance point on the pedicle (see H. N. Herkowitz, supra, at 93-94; and The Adult Spine, supra, at 1935). A screw opening is then formed in the pedicle using the disclosed awl, as generally depicted in FIG. 5. The surgeon advances the tip of the awl and views the reflected color displayed on the user interface device as illustrated in the embodiment depicted in FIG. 4. By noting color changes, the tip may be advanced through bone of both cancellous and cortical types, and any impingement on nerve tissue will be evident, as this will require repositioning of the bore. The awl is then withdrawn and a screw inserted. This procedure is repeated until the required number and arrangement of cortical screws have been inserted.

Although the lumbar spinal region of a patient is described above, the present screw insertion procedure is not limited in application to the lumbar region of the patient, or to the vertebrae, as will be appreciated by those skilled in the art.

Moreover, the present invention may be employed in robotic surgeries by connecting the awl of the present invention to a robotic actuator (arm) and providing the color signal to the surgeon, or to a controller system such as an artificial intelligence system adapted to guide the tool and to apply force thereto. Additionally, the present invention may be employed in minimally invasive surgical procedures by using a canulated awl and separate sensor probe that may be inserted through the awl to bring the illuminating and observing optical fibers into close proximity to the tissue at the tip of the awl. Alternatively a trocar may be used for softer tissues than hard bone, and the probe inserted through the trocar.

While the invention has been described in its preferred embodiments, it is to be understood that the words which have been used are words of description rather than of limitation and that changes may be made within the purview of the appended claims without departing from the true scope and spirit of the invention in its broader aspects. Rather, various modifications may he made in the details within the scope and range of equivalents of the claims and without departing from the spirit of the invention. The inventors further require that the scope accorded their claims be in accordance with the broadest possible construction available under the law as it exists on the date of filing hereof (and of the application from which this application obtains priority, if any) and that no narrowing of the scope of the appended claims be allowed due to subsequent changes in the law, as such a narrowing would constitute an ex post facto adjudication, and a taking without due process or just compensation.

What is claimed is:

1. A surgical tool system for forming openings in a patient's bone, the bone having nerve tissue in the region of the openings to be formed, comprising:
a tool handle shaped to be held and manipulated by a user when forming openings in the patient's bone;
a power source, illumination source, color sensor, microcontroller, and radio frequency transceiver, located inside the tool handle;
a shaft extending from a proximal end joined to the tool handle, to a distal end adapted to form openings in bone, the shaft containing an illuminating optical fiber having a proximal end optically coupled to the illumination source, and a sensing optical fiber having a proximal end optically coupled to the color sensor, each of the fibers having a distal end embedded at the distal end of the shaft; and
a display having a radio frequency transceiver for communicating with the radio frequency transceiver in the tool, the display showing the color of the tissue located adjacent the distal end of the shaft.

2. The surgical tool of claim 1 wherein the illumination source is a white light LED.

3. The surgical tool of claim 1 wherein the shaft is canulated.

4. The surgical tool of claim 1 wherein the shaft is further provided with a force sensor that is electrically coupled to the microcontroller.

5. A surgical system comprising a general purpose digital computer having a visual display and radio frequency communication capabilities, the computer running special purpose software stored in the memory of the computer, the software adapted to process and display data received via radio frequency transmission from a surgical tool having color sensing capability; a surgical tool comprising handle containing a power source, illumination source, color sensor, microcontroller, and a radio frequency transceiver; a shaft extending from a proximal end joined to the tool handle, to a distal end adapted to form openings in bone, the shaft containing an illuminating optical fiber having a proximal end optically coupled to the illumination source, and a sensing optical fiber having a proximal end optically coupled to the color sensor, each of the fibers having a distal end embedded at the distal end of the shaft.

6. A packaged sterile surgical kit comprising a tray, the tool of claim 1, and a sealing lid.

7. The tool of claim 1 wherein a unique serial number is displayed visually on the exterior of the tool, and is encoded electronically in the microcontroller's memory, and wherein the encoded serial number is transmitted via radio frequency to the display.

8. A method of forming an opening in bone for the insertion of a surgical appliance comprising the steps of
a) removing an insulating member to establish connection between a power source and a microcontroller circuit;
b) initiating a software program running on a general purpose digital computer, the software program adapted to process and display data received from a surgical tool having color sensing capability; the surgical tool comprising handle containing a power source, illumination source, color sensor, microcontroller, and a radio frequency transceiver; a shaft extending from a proximal end joined to the tool handle, to a distal end adapted to form openings in bone, the shaft containing an illuminating optical fiber having a proximal end optically coupled to the illumination source, and a sensing optical fiber having a proximal end optically coupled to the color sensor, each of the fibers having a distal end embedded at the distal end of the shaft;
c) impinging the surgical tool on the bone to be opened using sufficient force to create an opening in the bone;
d) observing the color displayed on the computer display and in response to the displayed color, changing the force applied to the tool, the position of the tool, or the angle of the tool.

9. The method of claim 8 wherein the impinging step c is facilitated by positioning and moving the tool using a surgical robot.

10. The method of claim 8 wherein the observing and force changing step d is performed using an autonomous artificially intelligent controller and actuator.
